(19) United States
(12) Patent Application Publication
(10) Pub. No.: US 2022/0354805 A1
(43) Pub. Date: Nov. 10, 2022

(54) Cannabidiol (CBD) as Chemical for Treating Aging-related Degenerative Diseases and Promoting Health Aging

(71) Applicant: Hemix Biotech Limited, Hong Kong (HK)

(72) Inventors: Sheung Kin Ken Wong, Hong Kong (HK); Zhongjun Zhou, Hong Kong (HK); Xuelai Wang, Hong Kong (HK); Jing Chen, Hong Kong (HK); Leung Kris Ying, Hong Kong (HK); Jian Zhang, Shanghai (CN)

(21) Appl. No.: 17/732,659

(22) Filed: Apr. 29, 2022

Related U.S. Application Data
(60) Provisional application No. 63/181,972, filed on Apr. 30, 2021.

(51) Int. Cl. A61K 31/05; A61K 36/185; A61K 38/17; C12N 5/077; G01N 1/30; A61P 25/28

(52) U.S. Cl. CPC A61K 31/05; A61K 36/185; A61K 38/1709; C12N 5/0656; G01N 1/30; A61P 25/28

(57) ABSTRACT

The invention relates to the technical field of medicine, and provides a method of screening and identifying small molecule with anti-aging properties. The method comprises cellular models, namely premature aging mouse embryonic fibroblasts and human HGPS mesenchymal stem cells and animal models, namely premature aging progeroid mice and Caenorhabditis elegans. The method successfully screened and identified Cannabidiol (CBD), which had senolytic effects. The invention also determined that the optimal concentration of CBD treatment is 10-20 μM.

### CROSS-REFERENCE TO RELATED APPLICATIONS

This application claims priority to U.S. Provisional Patent Application No. 63/181,972, entitled “Cannabidiol (CBD) as chemical for treating aging-related degenerative diseases and promoting health aging”, filed on Apr. 30, 2021, the disclosure of which is incorporated by reference in its entirety.

### BACKGROUND OF THE INVENTION

The present invention relates to the technical field of medicine. More particularly, the present invention is in the technical field of screening and identifying small molecules that have anti-aging properties.

Cannabidiol (CBD), a major non-psychotropic phytocannabinoid found in cannabis, has been used in treatments and clinical trials in various diseases including chronic pain, anorexia, nausea, spasticity and multiple sclerosis. Cannabis is also being used worldwide in treating a variety of skin conditions including acne, atopic dermatitis, psoriasis, skin cancer, pruritus, and pain. Cannabis also has been applied as an anti-aging supplement and skin care product.

US patent publication no. 20190216695 disclosed methods for lightening skin tone including topically administering a composition containing a cannabinoid, cannabidiol, cannabidiol analog, or combinations thereof. However, the said prior art reference did not disclose the method of screening and identifying the chemical and/or small molecules.

CN114272356 disclosed a formula of a pharmaceutical composition for delaying senility and a preparation method thereof. The said specific formula contains beta-glucan, chitosan, coenzyme Q10, CBD, glutathione, lipoic acid, lutein, nicotinamide, vitamin B2. However, this said prior art reference did not mention the method of screening and identifying the chemical and small molecules.

In view of the wide expansion of application scenarios of CBD, it is therefore desirable to have a quick and effective method to screen and identify the chemical and molecules having anti-aging effects. It is also desirable to have a study on its application on mammals.

The present invention provides a method of screening and identifying chemical and small molecules having anti-aging properties by using cellular models, namely the premature aging mouse embryonic fibroblasts and human HGPS mesenchymal stem cells and animal models, namely the premature aging progeroid mice and Caenorhabditis elegans. According to the embodiment of the invention, CBD was identified as an anti-aging chemical which had potential senolytic effects.

### SUMMARY OF THE INVENTION

It was known that Lamin A (LMNA)^{G609G/G609G} mutation mouse is a model of Hutchinson Gilford progeria syndrome (HGPS). HGPS is caused by point mutation in human LMNA which results in the production of truncated form of LMNA protein known as progerin. Expression level of progerin is also increased in old aged humans. HGPS causes premature aging in a variety of tissues therefore used as a model for study of aging and aging-related degenerative diseases.

According to the present invention, premature aging mouse embryonic fibroblasts (MEFs) and human mesenchymal stem cells (MSCs) are used for screening of chemicals and small molecules that can delay senescence, preliminary results are available in 2 to 3 weeks' time. Chemicals or small molecules with anti-aging properties are further tested using animal models, namely the Caenorhabditis elegans and premature aging mice. Caenorhabditis elegans is a type of roundworm that have been used heavily in aging studying for decades. Mainly because their relatively short lifespan, about 3 weeks. A large variety of chemicals or small molecules can be tested on C. elegans in a short period of time. For premature aging LMNA^{G609G/G609G} mutation mice, they lived for 4 to 6 months. The results of the effects of chemicals or small molecules on their healthspan can be acquired as soon as 2 to 3 months.

One embodiment of the invention utilizes 2 cell models and 2 animal models which can identify chemicals or small molecules that have anti-aging properties effectively. The whole process takes only 4 to 5 months. The identified anti-aging chemicals or small molecules can be used for clinical trial for aging-related degenerative diseases or supplements promoting healthy aging.

According to embodiments of this invention, Cannabidiol (CBD) is identified by us as an anti-aging chemical in mammal. Furthermore, the concentrations of CBD used in different embodiments have a large variation which ranges from 0.01 μM to 50 μM. It was known that that cells can react to different concentrations of CBD very differently. Low CBD concentrations treatment can have opposite effects on gene expressions compared to high CBD concentrations treatment. According to embodiment of the invention, the optimal concentration of CBD to maximize its beneficial effects on aging has been identified. The concentration of CBD that causes adverse effects has also been identified. The embodiment of the present invention identified the optimal concentration of using of CBD which provides the most beneficial effects on human.

According to a first aspect of the invention, a method of screening and identifying small molecules with anti-aging properties is provided.

According to a second aspect of the invention, the small molecule with antiviral properties is CBD.

According to a third aspect of the invention, the optimal concentration of said small molecule in treatment is 10-20 μM.

According to a forth aspect of the invention, the use of the small molecule as screened and identified in development of a medicament for treatment of metabolic disorders, aging-related degenerative diseases, hair loss and wound healing is provided.

According to a fifth aspect of the invention, the use of the small molecule as screened and identified for treatment of metabolic disorders, aging-related degenerative diseases, hair loss and wound healing is provided, by administrating said small molecule to mammals and regulate the expression of proteins.

According to a sixth aspect of the invention, the use of the small molecule as screened and identified for treatment of metabolic, aging-related degenerative diseases is provided, by administrating said small molecule to regulate the expression of proteins ATF5, CEBPα, and TRIB3.

Unless otherwise defined, all technical and/or scientific term used herein have the same meaning as commonly understood by one of ordinary skills in the art to which the invention pertains. Although methods and materials similar or equivalent to those described herein can be used in the practice or testing of embodiments of the invention, exemplary methods and/or materials are described below. In addition, the materials, methods, and examples are illustrative only and are not intended to be necessarily limiting.

### BRIEF DESCRIPTION OF DRAWINGS

The patent or application file contains at least one drawing executed in color.

Copies of this patent or patent application publication with color drawing(s) will be provided by the Office upon request and payment of the necessary fee.

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regards, the description taken with the drawings makes apparent to those skilled in the how embodiments of the invention may be practiced.

In the drawings:

FIG. 1 is a flowchart of method for screening, identifying, verifying chemicals and/or small molecules having antiviral functions and properties.

FIG. 2a-d illustrates the effect of CBD on proliferation of wildtype and LMNA^{G609G/G609G} MEFs. WT stands for wildtype MEFs while 609 stands for LMNA^{G609G/G609G} MEFs. (*stands for p-value is between 0.01 and 0.05, **stands for p-value is between 0.001 and 0.01, ***stands for p-value is between 0.0001 and 0.001. (a, b) Bar chart showing effect of 4-day CBD treatment on PDL of wildtype and LMNA^{G609G/G609G} MEFs respectively. (c, d) Bar chart showing effect of 8-day CBD treatment on PDL of wildtype and LMNA^{G609G/G609G} MEFs respectively.)

FIG. 3a-d illustrates the effect of CBD on senescence level of wildtype and LMNA^{G609G/G609G} MEFs. WT stands for wildtype MEFs while 609 stands for LMNA^{G609G/G609G} MEFs. (* stands for p-value is between 0.01 and 0.05, **stands for p-value is between 0.001 and 0.01, ***stands for p-value is between 0.0001 and 0.001. (a, b) Bar chart showing 4-day high dosage (50 μM) of CBD treatment significantly increased the senescence level of wildtype and LMNA^{G609G/G609G} MEFs respectively. (c, d) Bar chart showing 8-day optimal dosage (10 μM) of CBD treatment significantly decreased the senescence level of wildtype and LMNA^{G609G/G609G} MEFs respectively.)

FIG. 4 illustrates the effect of CBD on nuclear circularity of wildtype and LMNA^{G609G/G609G} MEFs. (WT stands for wildtype MEFs while 609 stands for LMNA^{G609G/G609G} MEFs. *stands for p-value is between 0.01 and 0.05, **stands for p-value is between 0.001 and 0.01, ***stands for p-value is between 0.0001 and 0.001. 12-day CBD treatment significantly increased the nuclear circularity of LMNA^{G609G/G609G} MEFs.)

FIG. 5 illustrates the effect of CBD on senescence markers in wildtype and LMNA^{G609G/G609G} MEFs. (WT stands for wildtype MEFs while 609 stands for LMNA^{G609G/G609G} MEFs. By Western blotting, 4-day CBD treatment significantly downregulated senescence markers: p21 and p16 in wildtype and LMNA^{G609G/G609G} MEFs. The β-actin acts as loading control.)

FIG. 6a-b illustrates the effect of CBD on lifespan of C. elegans healthspan of premature aging LMNA^{G609G/G609G} mice. (* stands for p-value is between 0.01 and 0.05, **stands for p-value is between 0.001 and 0.01, ***stands for p-value is between 0.0001 and 0.001. (a) C. elegans receiving 10 or 20 μM CBD treatment showed significant lifespan increase. For control group, n=30. For 10 μM CBD group, n=25. For 20 μM CBD group, n=15. (b) Mice receiving 50 mg/kg CBD twice a week starting from 2 months old had significantly increased healthspan with an increase of 11.2%. For control group, n=10. For treatment group, n=9.)

FIG. 7a-b illustrates the effect of CBD on colony formation capacity of bone marrow stromal cells of premature aging LMNA^{G609G/G609G} mice. (WT stands for wildtype mice while 609 stands for LMNA^{G609G/G609G} mice. *stands for p-value is between 0.01 and 0.05, **stands for p-value is between 0.001 and 0.01, ***stands for p-value is between 0.0001 and 0.001. (a) Crystal violet colony formation assay of bone marrow stromal cells isolated from wildtype, LMNA^{G609G/G609G} control and LMNA^{G609G/G609G} CBD treated mice. (b) Quantification of CBD crystal violet colony formation assay. CBD significantly rescued the decline of bone marrow stromal cells in premature aging LMNA^{G609G/G609G} mice.)

FIG. 8 illustrates the effect of CBD on progeroid features of skin of premature aging LMNA^{G609G/G609G} mice. (WT stands for wildtype mice while 609 stands for LMNA^{G609G/G609G} mice. Hematoxylin and eosin staining showed decrease of skin follicle density and width of skin fat layer in premature aging LMNA^{G609G/G609G} mice, which were rescued by CBD treatment.)

FIG. 9 illustrates the effect of CBD on senescence level in liver of premature aging LMNA^{G609G/G609G} mice. (WT stands for wildtype mice while 609 stands for LMNA^{G609G/G609G} mice. Senescence-associated beta-galactosidase staining showed increase of senescence level in liver of premature aging LMNA^{G609G/G609G} mice, which were rescued by CBD treatment.)

FIG. 10a-c illustrates the results of bioinformatic analysis of RNA sequencing of wildtype and LMNA^{G609G/G609G} MEFs receiving control or CBD treatment. (WT_C stands for wildtype MEFs with control treatment, WT_10 stands for wildtype MEFs with 10 μM CBD treatment, MUT_C stands for LMNA^{G609G/G609G} MEFs with control treatment, and MUT_10 stands for LMNA^{G609G/G609G} MEFs with 10 μM CBD treatment. (a) Venn diagrams showing the comparison of differentially expressed genes in wildtype and LMNA^{G609G/G609G} MEFs with control or CBD treatment. (b) Transcription factors: Activating transcription factor 5 (ATF5), CCAAT enhancer binding protein alpha (CEBPα), novel inhibitor of histone acetyltransferase repressor (NIR) and protein kinases: mitogen-activated protein kinase kinase kinase kinase 3 (MAP4K3) and tribbles pseudokinase 3 (TRIB3) identified by RNA sequencing as genes that showed differential expressions by CBD treatment in wildtype and LMNA^{G609G/G609G} MEFs. (c) Protein interaction network of ATF5, CEBPα, and TRIB3. Such network also includes well known aging associated genes including TP53, PTEN, PCNA, EP300 etc. This suggests the differential expressions of ATF5, α, and TRIB3 pathway caused by CBD treatment could contribute to its anti-aging effects.)

FIG. 11a-c illustrates the effect of CBD on levels of targets identified by RNA sequencing in skin of premature aging LMNA^{G609G/G609G} mice. (WT stands for wildtype mice while 609 stands for LMNA^{G609G/G609G} mice. (a) Immunofluorescence staining showed TRIB3 was downregulated in skin of premature aging LMNA^{G609G/G609G} mice, which was rescued by CBD treatment. (b) Immunofluorescence staining showed ATF5 was downregulated in skin of premature aging LMNA^{G609G/G609G} mice, which was rescued by CBD treatment. (c) Immunofluorescence staining showed CEBPα was downregulated in skin of premature aging LMNA^{G609G/G609G} mice, which was rescued by CBD treatment.)

FIG. 12a-b illustrates the results of senolytic effect of CBD in mesenchymal stem cells (MSCs) upon irradiation induced senescence. (Non-IR stands for non-irradiated MSCs while IR stands for irradiated MSCs. *stands for p-value is between 0.01 and 0.05, **stands for p-value is between 0.001 and 0.01, ***stands for p-value is between 0.0001 and 0.001. (a) Bar chart showing senolytic effect of CBD treatment on PDL of MSCs upon irradiation induced senescence. (b) By Western blotting, 6-hour 10 μM CBD treatment significantly downregulated levels of senescence marker p21, BCL-2 antiapoptotic family members BCL-w, BCL-xL and MCL-1 while upregulated TRIB3 in irradiated samples (β-actin acts as loading control) which contribute to CBD's senolytic effect. 6-hour 10 μM CBD treatment significantly upregulated level of p-akt in non-irradiated samples which also contribute to CBD's senolytic effect, where the akt acts as loading control.)

LIST OF ABBREVIATIONS

| | |
|---|---|
| CBD | Cannabidiol |
| TRIB3 | Tribbles pseudokinase 3 |
| MEFs | Mouse embryonic fibroblasts |
| PDL | Population doubling level |
| DMEM | Dulbecco's modified Eagle's medium |
| FBS | Fetal bovine serum |
| HGPS | Hutchinson Gilford progeria syndrome |
| PBS | Phosphate buffered saline |
| DEPC | Diethylpyrocarbonate |
| qPCR | Quantitative polymerase chain reaction |
| IP | Intraperitoneal injection |
| PEG | Polyethylene glycol |
| DMSO | Dimethyl sulfoxide |
| WT | Wild type |
| MUT | Lamin A G609G mutation |
| DAPI | 4′,6-diamidino-2-phenylindole |
| ATF5 | Activating Transcription Factor 5 |
| HEPES | 4-(2-hydroxyethyl)-1-piperazineethanesulfonic acid |
| MSCs | Mesenchymal stem cells |
| C. elegans | Caenorhabdits elegans |
| CEBPα | CCAAT enhancer binding protein alpha |
| NIR | Novel inhibitor of histone acetyltransferase repressor |
| MAP4K3 | Mitogen-activated protein kinase kinase kinase kinase 3 |
| RNA | Ribonucleic acid |
| EDTA | Ethylenediaminetetraacetic acid |
| DTT | Dithiothreitol |
| SDS | Sodium dodecyl sulfate |
| SDS-PAGE | Sodium dodecyl sulfate-polyacrylamide gel electrophoresis |
| PVDF | Polyvinylidene difluoride |
| PBST | Phosphate Buffered Saline with Tween ® 20 |
| PBSTr | PBS with 0.1% Triton X-100 |
| NGM | Nematode growth medium |
| LB | Lysogeny broth |
| α-MEM | Minimum Essential Medium Eagle - alpha modification |
| ddH₂O | Double-distilled water |
| RIN | RNA integrity number |
| cDNA | Complementary DNA |

### DETAILED DESCRIPTION OF THE INVENTION

The present invention will be further described below in connection with following embodiments. It should be understood that the following embodiments are intended to illustrate the invention only, but are not intended to limit the scope of protection of the present invention. Where specific conditions are not indicated in the following embodiments, they are performed according to conventional conditions or with reference to the manufacturer's protocols. The instruments or reagents used, where the manufacturer is not specified, are conventional products available commercially.

One embodiment of the invention utilizes premature aging LMNA^{G609G/G609G} mouse embryonic fibroblasts (MEFs), human mesenchymal stem cells (MSCs), Caenorhabditis elegansand premature aging LMNA^{G609G/G609G} mice to rapidly screen out anti-aging chemicals. LMNA G609G mutation mouse is a model of Hutchinson Gilford progeria syndrome (HGPS). HGPS is caused by a point mutation in human LMNA which results in producing a truncated form of LMNA protein known as progerin. The expression level of progerin is also increased in old-aged humans. HGPS causes premature aging in a variety of tissues therefore used as a model for the study of aging and aging-related degenerative diseases.

According to the embodiment of the invention, CBD was identified as an anti-aging chemical with senolytic effects through regulation of regulation of AKT and BCL-2 anti-apoptotic family pathways. Novel aging-associated genes including TRIB3, ATF5 and α were identified through the invention, which can be used as new gene targets for developing new anti-aging intervention. In brief, the outcome of this invention can promote healthy aging and provide a new treatment method for patients suffering various metabolic diseases, such as obesity, cardiovascular diseases, diabetes, neurodegenerative diseases, premature aging syndromes, aging, hair loss and wound healing.

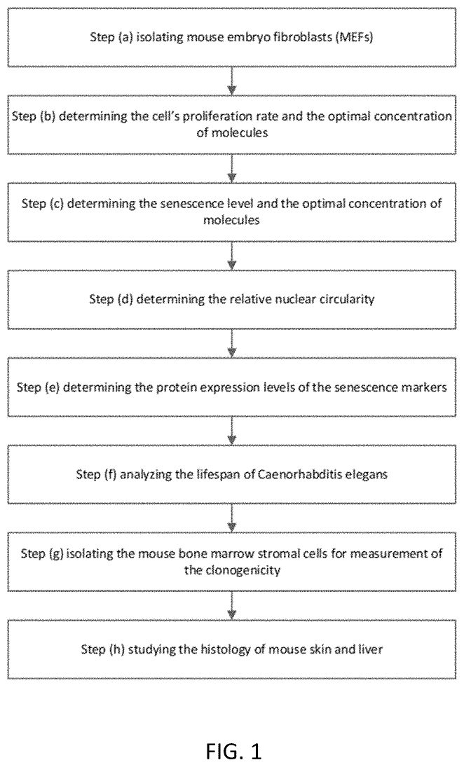

According to the embodiment of the present invention, a method of screening and identifying small chemicals with anti-aging functions and properties is provided. FIG. 1 is a flow diagram of a method of screening and identifying small molecules with anti-aging properties. Referring to FIG. 1, the method comprised of the steps of:
Step (a) isolating mouse embryo fibroblasts (MEFs) from heterozygous mice;
Step (b) determining the cell's proliferation rate and the optimal concentration of the small molecule by treating the MEFs of step (a) with the small molecule at different concentration, and calculating the PDL (population doubling level) of the cells;
Step (c) determining the senescence level of MEFs of step (a) and the optimal concentration of the small molecule by staining the MEFs of step (a), treating the said MEFs with the small molecule at different concentration, and then counting the number of stained and unstained cells;
Step (d) determining the relative nuclear circularity of MEFs of step (a) by treating with the small molecule at different concentration, and then measuring the percentage of cells in different nuclear circulatory level;
Step (e) determining the protein expression levels of senescence markers of the MEFs of step (a), by treating the cells with the small molecule, and then measuring the proteins' signals of the senescence markers, wherein the senescence markers are proteins p21 and p16;
Step (f) determining the optimal concentration of the small molecule by analyzing the lifespan of Caenorhabditis elegans under treatment of the small molecule at different concentration;
Step (g) determining the optimal concentration of the small molecule by analyzing the healthspan of LMNA^{G609G/G609G} mice under treatment of the small molecule at different concentration;
Step (h) verifying the anti-aging effect of the small molecule by isolating the bone marrow stromal cells from mouse and carrying out crystal violet staining colony formation assay; and Step (i) confirming the anti-aging effect of the small molecule by carrying out histology study of mouse skin and liver.

Using the method as described in FIG. 1, whether a small molecule has anti-aging function or properties can be quickly screened and identified. Materials and methods of the embodiment is described as below in detail:

### Step (a)—Isolation of Mouse Embryo Fibroblasts (MEFs)

In the step of isolating mouse embryo fibroblasts (MEFs), heterozygous (LMNA^{G609G/+}) mice were set for mating. The date of pregnancy of mice was identified by checking for vaginal plug in the morning following the day of mating. If the vaginal plug was found, the mouse was considered to be pregnant for 0.5 days (E0.5). When the embryos reached E12.5 to E13.5, the pregnant mice were sacrificed, and the embryos were isolated from the mice inside tissue culture hood using sterile utensils. The embryos were placed in phosphate buffered saline (PBS). Their head and liver were removed. A portion of the head was used for genotyping to identify the genotype of each embryo. The body of each embryo was transferred to 1mL of 0.1% Trypsin-EDTA solution in a well of 12-well plate. Using sterile scissors, embryos were cut into small pieces. The 12-well plate was then placed in a 37° C. incubator for 10 minutes. Followed by vigorous pipetting of the embryos in Trypsin-EDTA solution until the embryos wholly dissolved into the solution. The 12-well plate was then placed in 37° C. incubator for 5 minutes. The homogenized solution was then transferred to 9 mL of Gibco's High Glucose Dulbecco's Modified Eagle's Medium (DMEM) supplemented with sodium bicarbonate (3.7 g/L), HEPES (6 g/L), 10% fetal bovine serum (FBS) and penicillin-streptomycin (100 units/mL). The isolated MEFs were considered to be at passage 0 (P0). P3 MEFs were used in experiments as they have optimal cellular responses to stimuli. Primary mouse embryonic fibroblasts (MEFs) were cultured in Gibco's High Glucose Dulbecco's Modified Eagle's Medium (DMEM) supplemented with sodium bicarbonate (3.7 g/L), and 10% fetal bovine serum (FBS).

### Step (b)—Determination of Cell's Proliferation Rate and Optimal Concentration

In the step of determining the cell's proliferation rate and optimal concentration, primary mouse embryonic fibroblasts (MEFs) at passage 3 were counted by using LUNA-II Automated Cell Counter. 1.0×10⁵ cells were seeded to a well of 6-well plate. The cells were treated with DMSO as control, 10 μM or 50 μM CBD. After 4-day and 8-day treatment, the cells were counted again using LUNA-II Automated Cell Counter. The population doubling level (PDL) which reflects the proliferation rate of cells was calculated by the formula: n=3.32×(logUCY−logI)+X, where n=the PDL number, UCY=the cell yield at that time point, I=the initial cell number, and X=the doubling level of the cells used to initiate the subculture being quantitated.

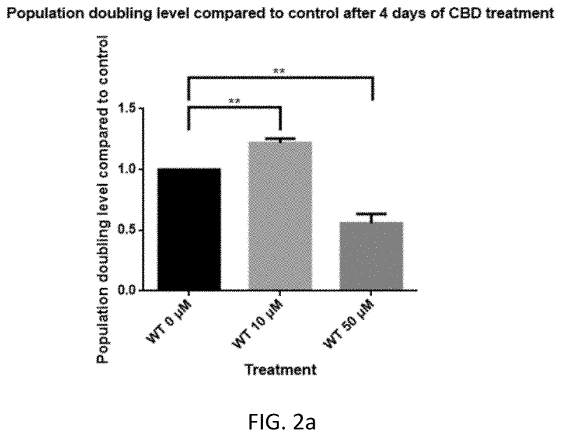

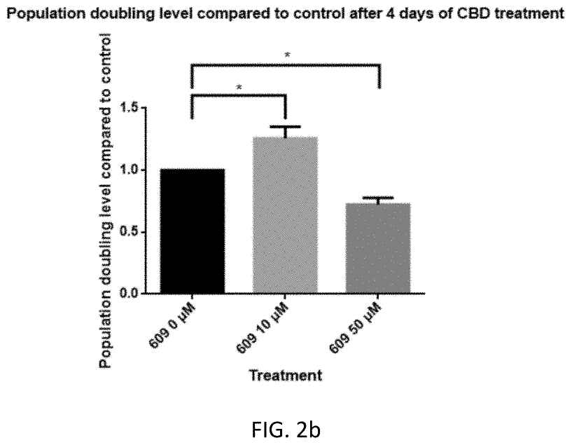

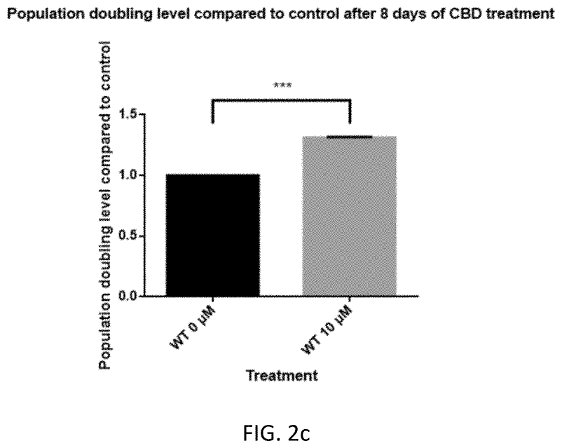

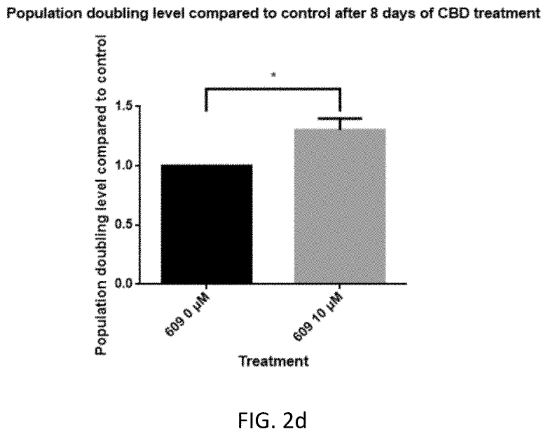

Referring to FIGS. 2a-d, for the cannabidiol (CBD) treatment, the optimal concentration was 10 μM which increased the PDL to the greatest extent after 4 and 8 days of treatment. Referring to FIGS. 2a-b, while 4 days of 50 μM CBD treatment led to a decrease in PDL which indicated toxicity effect due to high concertation.

### Step (c)—Determination of Senescence Level and Optimal Concentration

In the step of determining the senescence lever and optimal concentration, the primary mouse embryonic fibroblasts (MEFs) at passage 3 were seeded and cultured in chamber slides. The slides were washed twice with ice-cold phosphate buffered saline (PBS). Using senescence-associated beta-galactosidase staining kit, the cells were fixed for 10 minutes with provided paraformaldehyde and washed twice with PBS. The slides were then incubated with the staining solution at 37° C. overnight. On the following day, the slides were washed twice with PBS and mounted with 40% glycerol in PBS. The stained cells were observed under a light microscope. Senescent cells were stained blue while proliferating cells were unstained. The percentage of senescent cells was quantified by counting the number of stained and unstained cells.

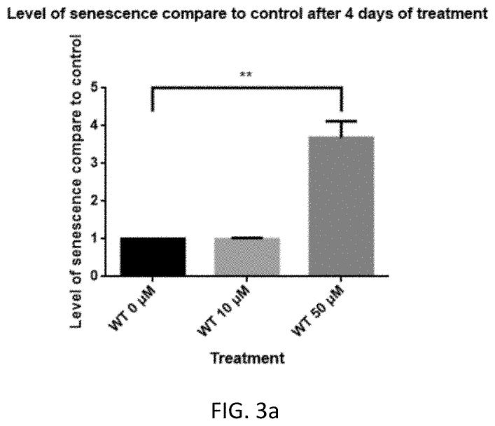

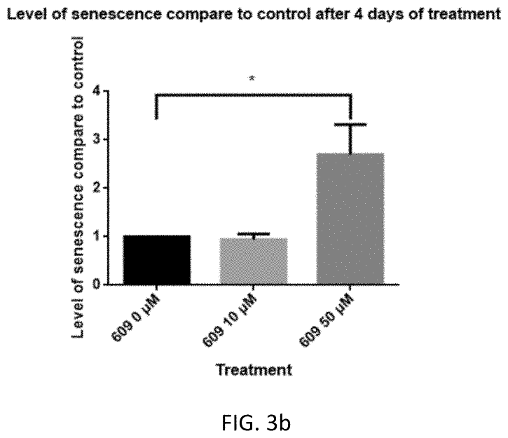

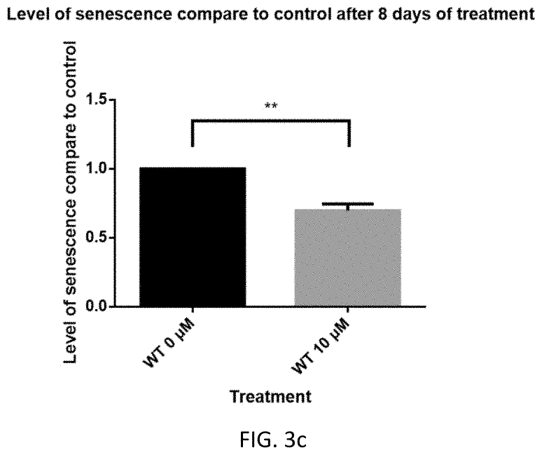

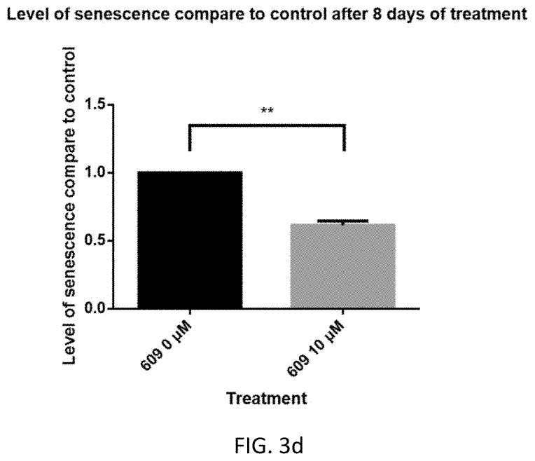

Referring to FIGS. 3c-d, by comparing CBD treated cells to control cells, 8 days of 10 μM CBD treatment showed decrease in senescence levels. Referring to FIGS. 3a-b, while 4 days of 50 μM CBD treatment showed increase in senescence levels which indicated toxicity effect due to high concertation.

### Step (d)—Determination of Relative Nuclear Circularity

In the step of determining the relative nuclear circularity, the primary mouse embryonic fibroblasts (MEFs) at passage 3 were seeded and cultured in chamber slides. Nuclei were stained using DAPI or LMNA/C antibodies. The fluorescence images were captured using confocal microscopy. The perimeter and area of the individual nucleus were measured using software CellProfiler. Relative nuclear circularity was calculated using the formula:

$$4 π \times ( \frac{area}{{perimeter}^{2}} ) .$$

A value of 1 in relative nuclear circularity indicates a perfect circle. Nuclei with relative nuclear circularity closer to 0 indicate they are more irregularly shaped.

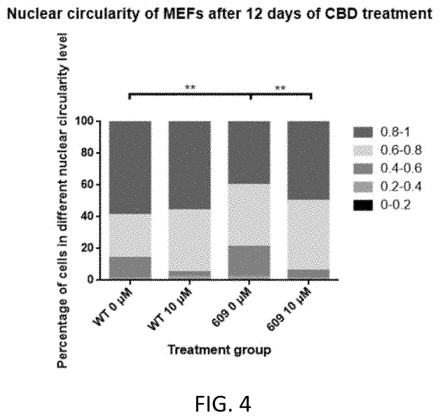

Referring to FIG. 4, the relative nuclear circularity was significantly decreased in LMNA^{G609G/G609G} MEFs compared to wildtype MEFs. While CBD treatment in LMNA^{G609G/G609G} MEFs significantly increased the relative nuclear circularity compared to control.

### Step (e)—Determination of Protein Expression Levels of Senescence Markers

In the step of determining the protein expression levels of senescence markers, the primary mouse embryonic fibroblasts (MEFs) at passage 3 were seeded and cultured in 6-well plate and treated with CBD for 4 days. The cells were then washed twice with ice-cold phosphate buffered saline (PBS). RIPA 150 buffer (20 mM Tris-HCl pH 7.5, 1 mM EDTA pH 8.0, 150 mM NaCl, 1% Nonidet P-40, 0.5% sodium deoxycholate) supplemented with 1 mM dithiothreitol (DTT) and proteinase inhibitors was added to the cells and shook at 4° C. for 15 minutes. The cells were then collected into Eppendorf tubes and centrifuged at 12000 rpm at 4° C. for 10 minutes. The supernatant was collected and 6×SDS sample buffer (20 mM Tris-HCl pH 7.5, 30% glycerol, 10% sodium dodecyl sulfate (SDS), 0.6 M DTT, 0.03% bromophenol blue) was added. The samples were boiled for 5 minutes. Then they were ready for sodium dodecyl sulfate-polyacrylamide gel electrophoresis (SDS-PAGE) for Western blotting analysis. Depending on the sizes of target proteins, 7-15% polyacrylamide separating gel and 4% polyacrylamide stacking gel were made. Samples and protein ladders were loaded onto the polyacrylamide gel. Sodium dodecyl sulfate-polyacrylamide gel electrophoresis (SDS-PAGE) was performed in running buffer (25 mM Tris-HCl, 190 mM glycine and 0.1% SDS) under constant voltage of 100V for 20-30 minutes until the samples and protein ladders reached the separating gel. By then, SDS-PAGE was set to run under constant voltage of 120V for about 1 hour, depending on the size of target proteins. The proteins in the separating gel were transferred to polyvinylidene difluoride (PVDF) membrane in transfer buffer (25 mM Tris-HCl, 190 mM glycine, and 20% methanol) at constant current 0.4 A for 1.5 hours on ice. The membranes were blocked with 5% skimmed milk in phosphate buffered saline Tween-20 solution (PBST): 1×PBS and 0.1% Tween-20 and shook at room temperature for 1 hour. Primary antibodies diluted in 5% skimmed milk in PBST were added to the membrane and shook at 4° C. overnight. The membranes were washed with PBST three times, shook at room temperature for 10 minutes each time. Secondary antibodies diluted in 5% skimmed milk in PBST were added to the membrane and shook at room temperature for 1-2 hours. The membranes were rewashed with PBST three times. Using SuperSignal™ West Pico PLUS Chemiluminescent Substrate, the proteins' signals were visualized using ChemiDoc Imaging System.

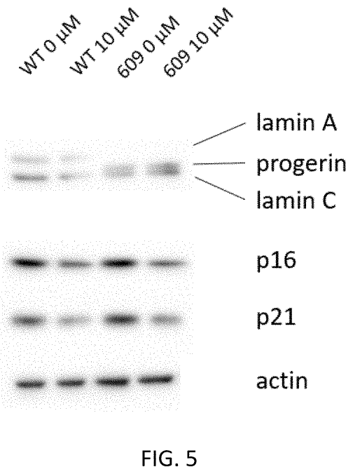

Referring to FIG. 5, it can be seen that CBD treatment could downregulate levels of p21 and p16 in both wildtype and LMNA^{G609G/G609G} MEFs while progerin levels were not affected. Therefore, CBD can downregulate p21 and p16 levels independent of progerin.

### Step (f)—Lifespan Analysis of Caenorhabditis elegans

In step of analyzing the lifespan of Caenorhabditis elegans, for chemical treatment in Caenorhabditis elegans, chemical was added to the nematode growth medium (NGM) dishes. The NGM with agarose was autoclaved and then cooled down to below 65° C. The different concentrations of chemical were added to NGM. The NGM was then poured into 60 mm dishes and incubated at room temperature overnight. The solidified NGM dishes were stored at 4° C. Escherichia coli OP50 strain was used as the food source for C. elegans. A single colony of OP50 was incubated in 200 mL of lysogeny broth (LB) medium at 37° C. overnight. The OP50 was then killed by incubation at 65° C. for minutes. The dead OP50 was spread throughout the surface of the NGM dishes. The NGM dishes with dead OP50 and CBD were stored at 4° C. for up to 2 weeks. The synchronization of C. elegans was carried out by the bleaching method. 20 L4 larvae were picked to each 60 mm dish with different concentrations of chemical and cultured at 20° C. They were monitored every day, and the number of living, dead and missing C. elegans were recorded. The C. elegans were transferred to fresh Petri dishes every 2 days to separate the target worms from their offspring and to ensure if they had enough food source. 10-20 μM of CBD was identified to be the optimal dosage for extending the lifespan of C. elegans.

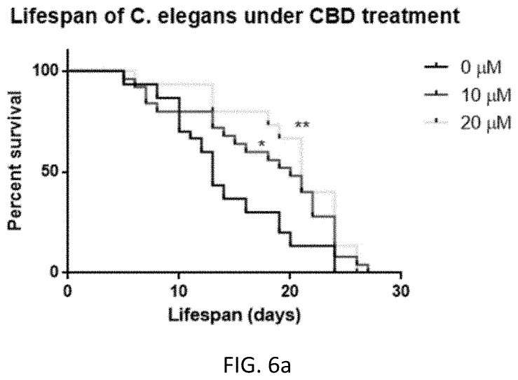

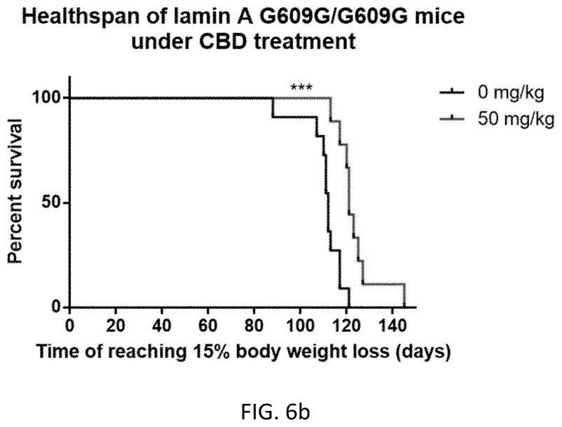

Referring to FIG. 6a, the median lifespan increase of CBD treated C. elegans was 55-61.5% while the maximum lifespan increase was 8-12.5%.

### Step (g)—Healthspan Analysis of LMNA^{G609G/G609G} Mice

In the step of analyzing the healthspan of LMNA^{G609G/G609G} mice, for chemical treatment in mice, 2 months old LMNA^{G609G/G609G} mice were used. They received an intraperitoneal injection (IP) twice a week until they reached the humane point. Different dosages were used for the injection, 50 mg/kg of CBD was identified as the optimal dosage with the greatest extend in healthspan of the treated mice. CBD was dissolved in 5% DMSO and 5% polyethylene glycol (PEG) in sterile saline and stored at −80° C. for up to 1 month.

Referring to FIG. 6b, there was an 8% and 19.8% increase in median and maximum healthspan of the CBD treated LMNA^{G609G/G609G} mice.

### Step (h)—Isolation of Mouse Bone Marrow Stromal Cells and Crystal Violet Staining Colony Formation Assay

In the step of isolating the mouse bone marrow stromal cells for measurement of the clonogenicity, mice were sacrificed and placed inside tissue culture hood. The femur was dissected out using sterile utensils. The mouse bone marrow stromal cells were flushed out of femur using a 29G needle with 1 mL of ice-cold phosphate buffered saline (PBS). The bone marrow stromal cells collected were centrifuged at 3000 rpm for 5 minutes at 4° C. The supernatant was removed, and the cell pellet was resuspended in Minimum Essential Medium Eagle—alpha modification (α-MEM) supplemented with 20% fetal bovine serum (FBS) and penicillin-streptomycin (100 units/mL). The medium was changed every 3 days. After 12 days of culture, crystal violet staining colony formation assay was carried out. The cells were washed twice with ice-cold PBS. The cells were fixed with ice-cold methanol for 10 minutes. Then the cells were stained with 0.5% crystal violet dissolved in 25% methanol for 10 minutes at room temperature. The crystal violet solution was then removed, and the cells were rinsed with double-distilled water (ddH₂O) until the rinse became clear. The plates were dried off, and pictures were taken for analysis.

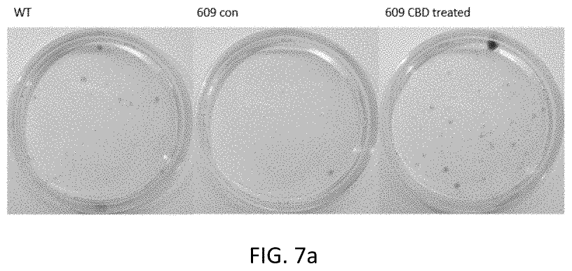

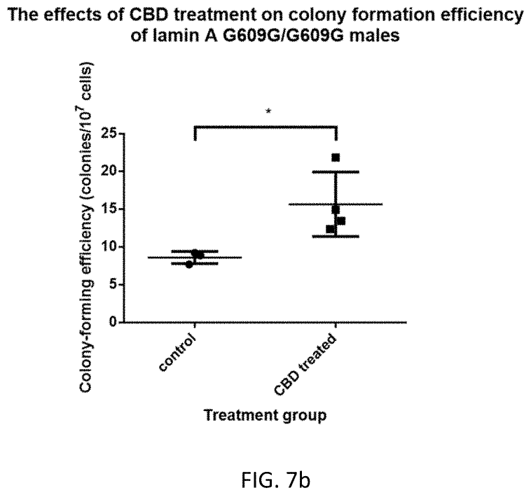

Referring to FIGS. 7a-b, the results showed that bone marrow stromal cells isolated from LMNA^{G609G/G609G} mice had a lower number of colonies compared to that of wildtype mice. While bone marrow stromal cells isolated from LMNA^{G609G/G609G} mice treated with 50 mg/kg of CBD had a higher number of colonies compared to LMNA^{G609G/G609G} control mice and wildtype mice.

### Step (i). Histology Study of Mouse Skin and Liver

In the step of study the histology of mouse skin and liver, the mice were sacrificed by cervical dislocation under anesthesia by intraperitoneal (IP) injection of 100 mg/kg ketamine and 16 mg/kg xylazine in sterile water. Major organs, including skin, muscle, intestine, kidney, spleen, liver, lung, heart, aorta, were collected. Half of the tissues were snap-frozen in liquid nitrogen and stored at −80° C. for extraction of proteins and RNA. Another half of the tissues were fixed in 4% paraformaldehyde (PFA) in PBS and shook at 4° C. overnight. For paraffin wax sections, the fixed tissues were incubated in 70% ethanol and shook at 4° C. overnight. Followed by stepwise dehydration of tissues in 90%, 95%, 100%, and 100% ethanol for 1 hour each. Then the tissues were incubated in 50% xylene and 50% ethanol for 30 to 40 minutes and transferred to 100% xylene for 10 to 15 minutes. The tissues were then incubated in wax for 30 minutes and transferred to new wax for 3 times. Then the tissues were placed in the wax inside a vacuum chamber overnight. On the following day, the tissues were embedded into paraffin blocks and sectioned into slides with 6 μm in thickness. After the tissue sections were dried, they were incubated in xylene solution twice each for 5 minutes. Stepwise rehydration was then carried out by incubating the tissue sections in 100%, 95%, 90%, 75%, 50%, and 30% ethanol each for 5 minutes and rinsed in water for 3 minutes. Hematoxylin and eosin staining was then carried out under standard procedure according to the manufacturer. Lastly, the stained tissues sections were mounted with DPX and dried overnight then observed under light microscope.

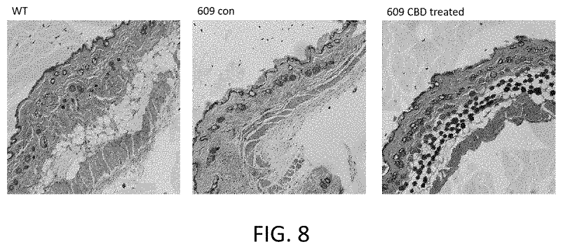

Referring to FIG. 8, it can be observed that the skin of LMNA^{G609G/G609G} showed progeroid features including loss of skin adipose layer and decrease of hair follicle density which could be rescued by CBD treatment. For senescence staining, senescence-associated beta-galactosidase staining kit was used after stepwise rehydration of the tissues.

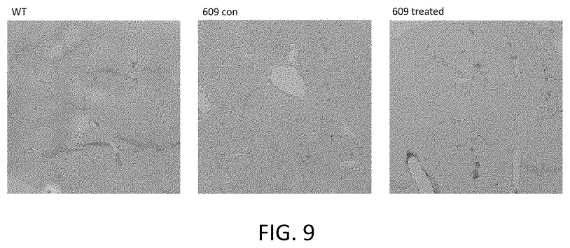

Referring to FIG. 9, it can be observed that the liver of LMNA^{G609G/G609G} mice showed increase of senescent cells which could be rescued by CBD treatment.

It can be seen from the above embodiments, the present invention used the method of screening and identifying small molecules with anti-aging properties, have successfully screened and identified CBD, a small molecule with anti-aging properties. It is also determined that the optimal dosage of CBD treatment is 10-20 μM.

Any of the features, attributes, or steps of the above described embodiments and variations can be used in combination with any of the other features, attributes, and steps of the above described embodiments and variations as desired.

According to the embodiment of the present invention, CBD was chosen to verify the anti-aging effects. The effects have been verified based on various modules. Also, the mechanism behind the anti-aging effects of the small chemicals is identified by using various methods. Materials and methods of the embodiment is described as below in details:

### Identification of Differentially Expressed Genes Through RNA Sequencing and Validation Through Quantitative Polymerase Chain Reaction (qPCR)

Primary mouse embryonic fibroblasts (MEFs) at passage 3 were seeded and cultured in 10-cm dish and treated with CBD for 4 days. The cells were washed twice with ice-cold phosphate buffered saline (PBS), and 1 mL TRIzol was added. After incubation at room temperature for 5 minutes, the samples were collected into Eppendorf tubes. The supernatants were collected to Eppendorf tubes. 200 μl of chloroform was added to each sample, and the tubes were shaken vigorously by hand for 15 seconds. The samples were incubated at room temperature for 5 minutes and then centrifuged at 4° C. at 12000 rcf for 15 minutes. After centrifugation, the solution was separated into 3 layers. 450 μl of the clear top layer was collected, and 500 μl of isopropanol was added. After incubation at room temperature for 10 minutes, the samples were centrifugated at 4° C. at 12000 rcf for 10 minutes. RNA pellets would be visible at the bottom of the tubes. The RNA pellets were washed by 75% ethanol in diethylpyrocarbonate (DEPC) water. The RNA in 75% ethanol can be stored at −20° C. for 1 year. For each condition, two biological replicates were sent for RNA sequencing. The RNA samples were dissolved in DEPC water and carried out quality control analysis. The RNA quantitation was done using Nanodrop and Agilent 2100 Bioanalyzer. The RNA integrity was measured using Agilent 2100 Bioanalyzer. The RNA purity was measured using agarose gel electrophoresis and Agilent 2100 Bioanalyzer. The samples that had concentration over 50 ng/μl for volume over 20 μl, RIN value over 6.3, and OD260/280 value over 2.0 were considered to pass the quality control analysis and were used for RNA sequencing.

Illumina sequencing system was used for paired-end reads of read lengths PE150. Bioinformatic analysis was performed, which included data quality control, alignment, gene expression level analysis, differential gene expression analysis, and functional analysis. The results were validated by quantitative polymerase chain reaction (qPCR). For RNA using for qPCR, the samples were centrifugated at 4° C. at 7500 rcf for 5 minutes. The ethanol was removed, and the samples were air-dried until the RNA pellets turned transparent. Then the RNA pellets were resuspended with DEPC water. The concentration of RNA was measured by nanodrop. 2 μg of RNA was used for DNase digestion and reverse transcription, while the rest can be stored at −80° C. for one month. For DNase digestion, 2 μg of RNA was used for each reaction using Promega RQ1 Rnase-Free Dnase following the manufacturer's protocol. DNase digestion removes the DNA contaminant in the RNA samples, which prevents the amplification of genomic DNA in qPCR. The DNase digested RNA samples were then used for reverse transcription using Thermo Scientific's High Capacity cDNA Reverse Transcription Kit with RNase Inhibitor following manufacturer's protocol. The transcribed cDNA was diluted 5 times with Milli-Qwater. Then the samples were ready for qPCR. The unused samples were stored at −20° C. For qPCR 0.5-1 μl of cDNA was used for each 10 μl reaction. The delta-delta-ct value was calculated by the qPCR machine based on the signal of target genes normalized with housekeeping genes.

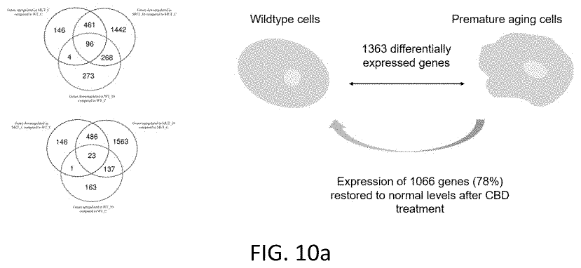

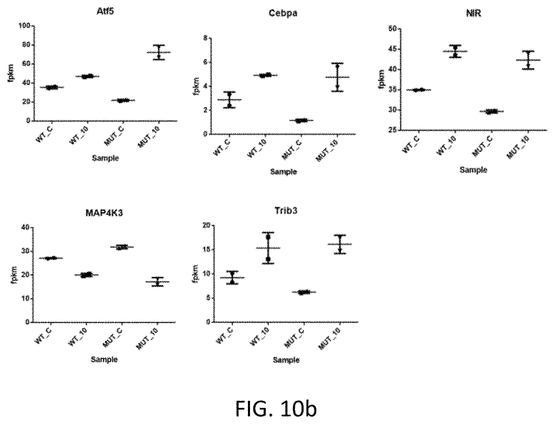

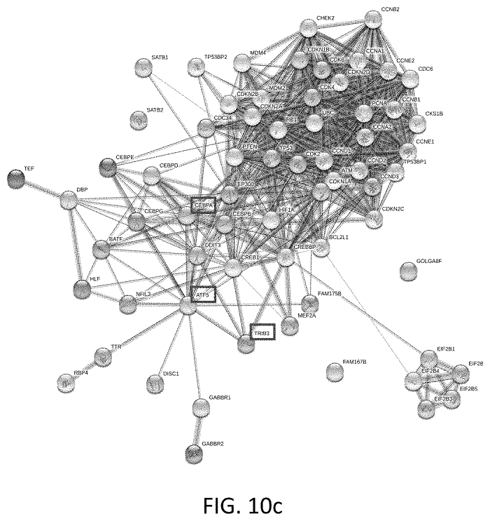

Referring to FIG. 10a, it can been seen that the RNA sequencing results of CBD treated MEFs revealed that expression of 1066 out of 1363 differentially expressed genes in LMNA^{G609G/G609G} MEFs restored to normal levels after CBD treatment, which was 78% of the differentially expressed genes. As a large number of genes were differentially expressed upon CBD treatment, it was shown that it was the result of differential expression of transcription factors or proteins affecting transcription activities. In the upper Venn diagrams of FIG. 10a, the upper-left circle of the stands for genes upregulated in MUT_C compared to WT_C, the upper-right circle stands for genes downregulated in MUT_10 compared to MUT_C, the lower circle stands for genes downregulated in WT_10 compared to WT_C. In the bottom Venn diagrams of FIG. 10a, the upper-left circle of the stands for genes downregulated in MUT_C compared to WT_C, the upper-right circle stands for genes upregulated in MUT_10 compared to MUT_C, the lower circle stands for genes upregulated in WT_10 compared to WT_C.

Referring to FIG. 10b, under such criteria and going through each gene, 5 genes were identified. They were transcription factors: activating transcription factor 5 (ATF5), CCAAT enhancer binding protein alpha (α), novel inhibitor of histone acetyltransferase repressor (NIR) and protein kinases: mitogen-activated protein kinase kinase kinase kinase 3 (MAP4K3) and tribbles pseudokinase 3 (TRIB3).

Referring to FIG. 10c, by comparing the protein interaction profile of each gene, ATF5, CEBPα, and TRIB3 were found to be closely related in the same protein interaction network. Such a network also includes genes that are associated with senescence, DNA damage, proliferation like ATM, PTEN, p53 53BP1, p300, etc. Therefore, the anti-aging properties of CBD could be partly contributed by the differential expression of ATF5, CEBPα, and TRIB3 in this protein interaction network.

### Immunofluorescence (IF) Staining of Tissues

The tissue sections were washed twice with PBS and incubated in PBSTr (0.1% Triton X-100 in PBS) for 10 minutes. Then the cells were blocked with 5% fetal bovine serum (FBS) in PBSTr for 1 hour at room temperature. Primary antibodies diluted in 5% FBS in PBSTr were added and incubated at 4° C. overnight. On the following day, the cells were washed three times using PBSTr, incubation for 10 minutes each time. Secondary antibodies diluted in 5% FBS in PBSTr were added and incubated at room temperature for 1 hour. The cells were again washed three times using PBSTr, followed by PBS for two times. The slides were mounted with SlowFade Gold antifade reagent. Then the slides were ready to be analyzed under a confocal microscope.

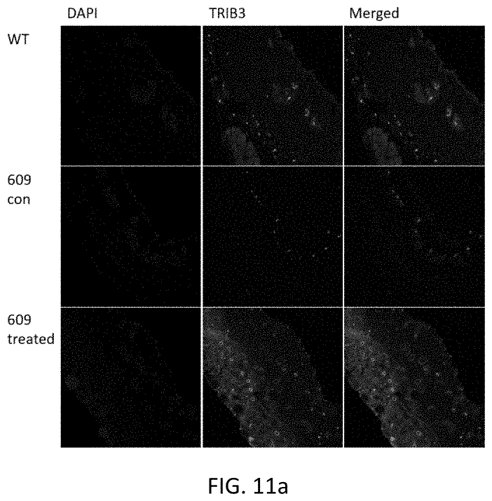

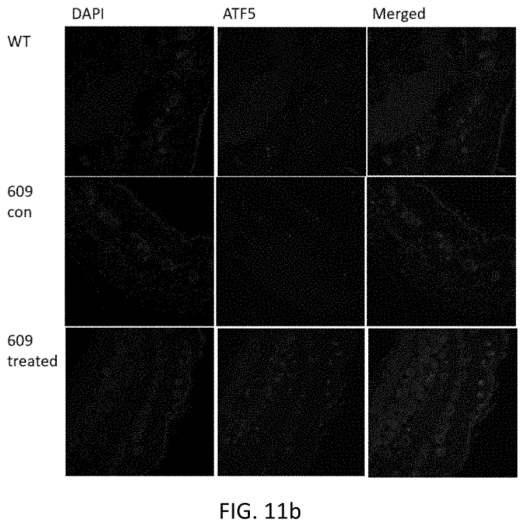

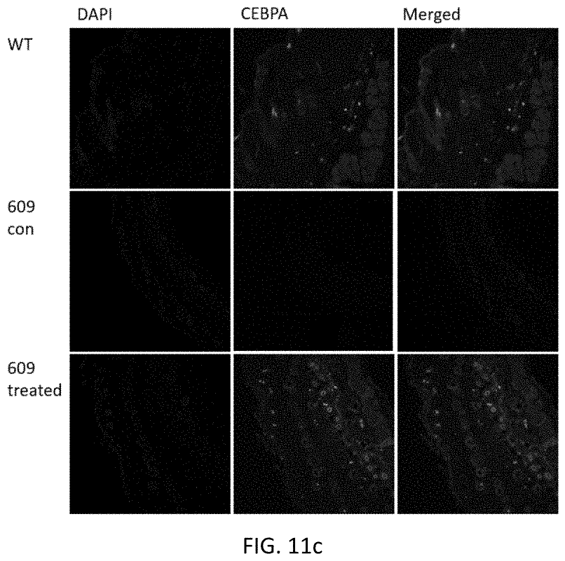

Referring to FIG. 11a-c, according to the results of immunofluorescence staining in mice skin, protein levels of ATF5, CEBPα and TRIB3 all decreased in premature aging mice, which all are reversed by CBD treatment. Therefore, these 3 proteins could be important aging-associated proteins.

### Examination of the Senolytic Effect of Chemicals

Human mesenchymal stem cells (MSCs) were counted by using LUNA-II Automated Cell Counter. 1.0×10⁵ cells were seeded to each well of 6-well plate and cultured for 24 hours. The cells were divided into two groups: non-irradiated and irradiated group. The irradiated group received gamma irradiation by irradiator. 10 Gy was the dosage of irradiation used. 24 hours after the irradiation, the non-irradiated and irradiated group were treated with different concentration of chemical. After 24-hour treatment, the cells were counted again using LUNA-II Automated Cell Counter. The population doubling level (PDL) which reflects the proliferation rate of cells was calculated by the formula: n=3.32×(logUCY−logI)+X, where n=the PDL number, UCY=the cell yield at that time point, I=the initial cell number, and X=the doubling level of the cells used to initiate the subculture being quantitated.

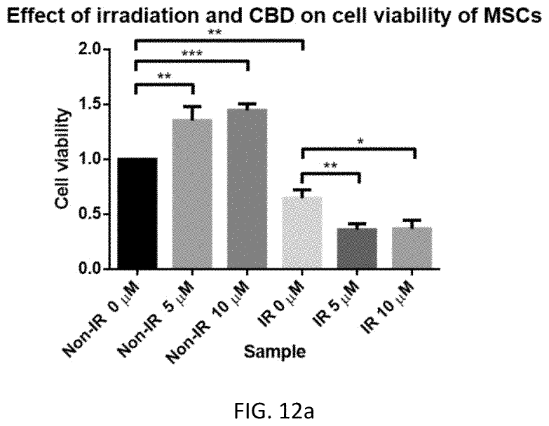

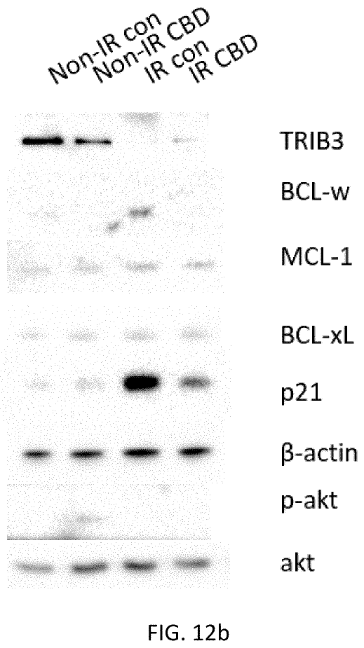

The relative cell viability was calculated by normalizing each sample to non-irradiated control sample. Referring to FIG. 12a, it was shown that that CBD treatment could significantly increase the cell viability of non-irradiated control MSCs while significantly decrease the cell viability of irradiated senescent MSCs. As CBD selectively killed senescent MSCs while keeping proliferating MSCs unharmed, it was discovered to be a novel senolytic. Referring to FIG. 12b, Western blotting was carried to investigate the mechanism behind CBD's senolytic effect. Anti-apoptotic BCL2 family proteins including BCL-w, MCL-1 and BCL-xL were found to be downregulated. While as proliferating and senescent MSCs responded differently in phosphorylated akt level. Expression level of TRIB3 was downregulated in senescent MSCs while was rescued by CBD treatment. Therefore, it was demonstrated that that CBD could promote apoptosis in senescent MSCs and therefore results in senolytic effect.

In conclusion, the present invention herein provided that Cannabidiol (CBD) can delay cellular senescence in mouse embryonic fibroblasts (MEFs). Firstly, it is showed that CBD can increase the replication rate of wildtype and premature aging LMNA^{G609G/G609G} MEFs. Secondly, CBD can decrease the senescence level of wildtype and premature aging LMNA^{G609G/G609G} MEFs. Thirdly, CBD can rescue the misshaped nucleus phenotype of premature aging LMNA^{G609G/G609G} MEFs. Fourthly, CBD can downregulate the protein level of senescence markers: p21 and p16. Fifthly, CBD can increase the lifespan of C. elegans and the healthspan of premature aging LMNA^{G609G/G609G} mice. Sixthly, CBD can rescue the decline of bone marrow stromal cells, increase skin hair follicle density, and increase skin fat layer width in LMNA^{G609G/G609G} mice. Seventhly, CBD showed senolytic effects in mesenchymal stem cells (MSCs) upon irradiation induced senescence through regulation of AKT and BCL-2 anti-apoptotic family pathways. CBD treatment killed senescent cells while keeping non-senescent cells unharmed. Lastly, transcription factors, namely activating transcription factor 5 (ATF5), CCAAT enhancer binding protein alpha (CEBPα), novel inhibitor of histone acetyltransferase repressor (NIR) and protein kinases, namely mitogen-activated protein kinase kinase kinase kinase 3 (MAP4K3) and tribbles pseudokinase 3 (TRIB3) were identified by RNA sequencing as genes that showed differential expressions by CBD treatment in wildtype and LMNA^{G609G/G609G} MEFs. They all involve in gene pathways that are associated to aging. ATF5, CEBPα, and TRIB3 are found to be closely regulated in the same protein interaction network. Such network also includes well known aging associated genes including TP53, PTEN, PCNA, EP300 etc. The effects of CBD treatment on their expression levels may contribute to anti-aging effects. According to the results of immunofluorescence staining in mice skin. Protein levels of ATF5, CEBPα and TRIB3 all decreased in premature aging LMNA^{G609G/G609G} mice, which all are reversed by CBD treatment. Furthermore, methods are provided for the treatment of patients suffering various metabolic diseases, such as obesity, cardiovascular diseases, diabetes, neurodegenerative diseases, premature aging syndromes, aging, hair loss and wound healing.

Although the invention has been shown and described with respect to a certain embodiment or embodiments, it is apparent that this invention can be embodied in many different forms and that many other modifications and variations are possible without departing from the spirit and scope of this invention.

Moreover, while exemplary embodiments have been described herein, one of ordinary skill in the art will readily appreciate that the exemplary embodiments set forth above are merely illustrative in nature and should not be construed as to limit the claims in any manner. Rather, the scope of the invention is defined only by the appended claims and their equivalents, and not, by the preceding description.

The invention claimed is:

1. A method of screening and identifying small molecule which having anti-aging properties, the method comprising the following steps:
(a) isolating mouse embryo fibroblasts (MEFs) from heterozygous mice;
(b) determining the cell's proliferation rate and the optimal concentration of the small molecule by treating the MEFs of step (a) with the small molecule at different concentration, and then calculating the PDL (population doubling level) of the cells;
(c) determining the senescence level of MEFs of step (a) and the optimal concentration of the small molecule by staining the MEFs of step (a), treating the said MEFs with the small molecule at different concentration, and then counting the number of stained and unstained cells;
(d) determining the relative nuclear circularity of the MEFs of step (a) after treatment with the small molecule at different concentration by measuring the percentage of said cells in different nuclear circulatory level;
(e) determining the protein expression levels of senescence markers of the MEFs of step (a), by treating the cells with the small molecule, and then measuring the proteins' signals of the senescence markers, wherein the senescence markers are proteins p21 and p16;
(f) determining the optimal concentration of the small molecule by analyzing the lifespan of Caenorhabditis elegansunder treatment of the small molecule with different concentration;
(g) determining the optimal concentration of the small molecule by analyzing the healthspan of LMNA^{G609G/G609G} mice under treatment of the small molecule with different concentration;
(h) verifying the anti-aging effects of the small molecule by isolating the bone marrow stromal cells from mice, and then carrying out crystal violet staining colony formation assay; and
(i) confirming the anti-aging effects of the small molecule by carrying out histology analysis on mouse skin and liver.

2. The method according to claim 1 wherein said smaller molecule is Cannabidiol (CBD).

3. The method according to claim 2 where optimal concentration of said small molecule in treatment is 10-20 μM.

4. The use of the small molecule as screened and identified according to claim 1 in development of a medicament for treatment of metabolic disorders, aging-related degenerative diseases, hair loss and wound healing, by administration of said small molecule to mammals and regulation of the expression of proteins.

5. The use according to claim 4, wherein the smaller molecule is Cannabidiol (CBD).

6. The use according to claim 4, wherein the proteins are ATF5, CEBPα, or TRIB3.

7. The use according to claim 4, wherein the optimal concentration of said small molecule in the treatment is 10-20 μM.
